(19) United States
(12) Patent Application Publication
(10) Pub. No.: US 2024/0094618 A1
(43) Pub. Date: Mar. 21, 2024

(54) Low Profile Optical Systems for Surgical Procedures

(71) Applicant: Alcon Inc., Fribourg (CH)

(72) Inventors: Douglas Allen, Santa Barbara, CA (US); Eric Aspnes, Santa Barbara, CA (US)

(21) Appl. No.: 18/464,074

(22) Filed: Sep. 8, 2023

Related U.S. Application Data
(60) Provisional application No. 63/408,741, filed on Sep. 21, 2022.

(51) Int. Cl. G03B 35/10; A61B 90/00; G02B 15/14

(52) U.S. Cl. CPC G03B 35/10; A61B 90/361; G02B 15/14

(57) ABSTRACT

The present disclosure generally relates to optical systems for surgical procedures, and more particularly, to optical relay systems for visualization systems used during ophthalmic microsurgical procedures. The optical systems described herein provide improved ergonomics for surgeons, as such systems facilitate a low-height microscope camera that enables a surgeon to see over the camera to a display screen or other monitor placed in an ergonomically advantageous position for ophthalmic procedures. Such optical systems also provide improved performance, as the total magnification of the optical head may be split between multiple lens barrels, thereby creating high resolution images that enables surgeons to see ophthalmic anatomies more clearly. Even further, such optical systems proffer improved manufacturability, with reduced weight and manufacturing cost, as the lens barrels may have fixed focal lengths for utilization with digital magnification mechanisms.

### BACKGROUND

Vitreoretinal and cataract surgical procedures are among the most commonly-performed ophthalmic surgical procedures. As the name implies, vitreoretinal procedures are performed in the gel-like vitreous and on surfaces of the light-sensitive retina within the relatively small ocular space. Common conditions necessitating vitreoretinal surgery include epimacular membranes, vitreomacular schisis, vitreomacular traction syndrome, diabetic traction retinal detachments, proliferative vitreoretinopathy (PVR), retinal detachment, macular holes, as well as various micro-injection procedures for gene and cell based therapies. Cataract surgeries, on the other hand, involve the removal of a cloudy natural lens of the eye, and replacing it with a new artificial lens. There are two types of cataract surgeries: phacoemulsification, wherein the cloudy lens is removed after being broken up by application of ultrasound waves, and extracapsular surgery, wherein the cloudy core of the lens is removed in one piece.

Traditionally, surgeons performing vitreoretinal and/or cataract surgeries utilized surgical microscope eyepieces to provide magnified and illuminated images of ophthalmic structures. More recently, heads-up digital surgical visualization systems have started to replace the use of such microscopes. Such visualization systems, which rely on high-resolution stereoscopic cameras to transmit images from the patient's eye to a heads-up display screen for viewing by the surgeon, offer the advantages of better ergonomics for the surgeon, reduced phototoxicity, peripheral visualization, improved magnification, and less asthenopia as compared to traditional miscopies.

However, despite the many advantages of heads-up digital surgical visualization systems, current designs for the optical heads (i.e., the bodies carrying the optical components) used therewith are not optimized for ophthalmic surgeries in terms of optics, ergonomics, and/or general use. For example, current optical heads for such systems are tall and bulky, thereby causing them to impede surgical access to the patient's eye, as well as obstruct the surgeon's and/or assistant's view of the heads-up display screen. Even further, the optics of such optical heads introduce many optical distortions when increasing magnification or changing the field and/or depth of view, thereby decreasing the accuracy and precision of ophthalmic surgical procedures.

Accordingly, there is a need in the art for improved optical systems for visualization systems used during ophthalmic surgical procedures.

### SUMMARY

Embodiments of the present disclosure generally relate to optical systems for surgical procedures, and more particularly, to optical relay systems for visualization systems used during ophthalmic microsurgical procedures.

In certain embodiments, an optical system for a surgical camera is provided. The optical system includes: two stereoscopic channels, each stereoscopic channel having: a first lens barrel with one or more first optics, the first lens barrel configured to produce an image having a wide field of view (FOV); and a second lens barrel with one or more second optics, the second lens barrel configured to produce an image having a narrow FOV, wherein an optical load of each stereoscopic channel is split between the first lens barrel and the second lens barrel.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates a perspective view of an exemplary ophthalmic suite for ophthalmic surgical procedures, including a digital visualization system, according to certain embodiments of the present disclosure.

FIG. 2A illustrates a simplified top perspective view of an exemplary optical head, according to certain embodiments of the present disclosure.

FIG. 2B illustrates a bottom perspective view of the components of the optical head in FIG. 2A, according to certain embodiments of the present disclosure.

FIG. 2C illustrates another top perspective view of the components of the optical head in FIG. 2A, according to certain embodiments of the present disclosure.

FIG. 2D illustrates a front view of the components of the optical head in FIG. 2A, according to certain embodiments of the present disclosure.

FIG. 2E illustrates a side view of the components of the optical head in FIG. 2A, according to certain embodiments of the present disclosure.

FIG. 3A illustrates a simplified cross-sectional front view of an exemplary optical module, e.g., the optical module of the optical head in FIGS. 2A-2E, according to certain embodiments of the present disclosure.

FIG. 3B illustrates a rear perspective view of the optical module FIG. 3A, according to certain embodiments of the present disclosure.

FIG. 4A illustrates a perspective side view of an exemplary tandem of lens barrels of the optical module of FIG. 3A, according to certain embodiments of the present disclosure.

FIG. 4B illustrates a perspective side view of another exemplary tandem of lens barrels of the optical module of FIG. 3A, according to certain embodiments of the present disclosure.

FIG. 5A illustrates a schematic cross-sectional side view of a portion of an exemplary optical module, e.g., the optical module of the optical head in FIGS. 2A-2E, according to certain embodiments of the present disclosure.

FIG. 5B illustrates a schematic plan view of an exemplary configuration of the optical module of FIG. 5A, according to certain embodiments of the present disclosure.

FIG. 5C illustrates a schematic plan view of another exemplary configuration of the optical module of FIG. 5A, according to certain embodiments of the present disclosure.

FIG. 6A illustrates a perspective view of an exemplary configuration of a plurality of optical modules within an optical head for use during different types of ophthalmic procedures, according to certain embodiments of the present disclosure.

FIG. 6B illustrates a plan view of the exemplary optical head configuration of FIG. 5A, according to certain embodiments of the present disclosure.

FIG. 7A illustrates a patient's eye as viewed by the digital visualization system described herein, at different stages of magnification, according to certain embodiments of the present disclosure.

FIG. 7B illustrates a schematic cross-sectional side view of an exemplary optical head, e.g., the optical head in FIGS. 2A-2E, wherein an optical module thereof is moved to a first position, according to certain embodiments of the present disclosure.

FIG. 7C illustrates another schematic cross-sectional side view of the optical head in FIG. 7B, wherein the optical module thereof is moved to a second position, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end or portion of a component refers to the end or the portion that is closer to a patient's body during use thereof. On the other hand, a proximal end or portion of the component refers to the end or the portion that is distanced further away from the patient's body.

As used herein, the term “about” may refer to a +/−10% variation from the nominal value. It is to be understood that such a variation can be included in any value provided herein.

The present disclosure relates to optical systems for surgical procedures, and more particularly, to optical relay systems for visualization systems used during ophthalmic microsurgical procedures.

As described above, many current optical heads for heads-up digital surgical visualization systems are not optimized for ophthalmic surgeries in terms of optical components, ergonomics, and/or general use. For example, such optical heads may be tall and bulky, thereby causing them to impede surgical access to the patient's eye, as well as obstruct the surgeon's and/or assistant's view of the heads-up display screen. Even further, the optics in such optical heads may introduce optical distortions when increasing magnification or changing the field and/or depth of view, thereby decreasing the accuracy and precision of ophthalmic surgical procedures. The present disclosure addresses these and other shortcomings of many current designs by providing a robust and low-profile optical head for an ophthalmic 3D stereo microscope camera, which facilitates improved ergonomics and visibility during ophthalmic surgical procedures.

In certain embodiments, the optical head comprises an optical module having two stereo channels with two lens barrels per channel (i.e., a tandem of lens barrels per channel), the optical head thus having a total of four lens barrels. In other embodiments, the optical head comprises two or more optical modules, wherein each optical module includes two stereo channels each having two lens barrels, the optical head thus having a total of eight or more lens barrels. In such embodiments, each optical module may be optimized for performance of a different ophthalmic surgical procedure, e.g., cataract/anterior procedures or retinal/posterior procedures. For example, different optical modules may be more optimized than others to different depths-of-field in order to allow for anatomies at different depth levels along an optical axis of the microscope camera to be viewed more efficiently, and/or different optical modules may be more optimized than others to different resolutions depending on the size of anatomies that are desired to be viewed during an ophthalmic surgical procedure. In further embodiments, different optical modules may be configured to perform at different fields of view (e.g., magnification levels). In certain embodiments, different modules may also be coupled or utilized with different light filters.

In still further embodiments, each stereo channel of an optical head may be comprised on a submodule of an optical module. For example, in certain embodiments, an optical module may comprise a “left” and a “right” submodule corresponding to a “left” and “right” stereo channel, respectively. During assembly of the optical head, each submodule and thus, each stereo channel, may be built up and separately aligned to each other and other components of the optical head, thereby facilitating more efficient alignment and assembly of the optical head.

In certain embodiments, an optical head further comprises one or more coaxial illumination sources. The coaxial illumination sources may be mounted to the optical module(s) of the optical head, or other components of the optical head, such that illumination light produced by the coaxial illumination sources is coaxially aligned with one or more optical axes of the optical module(s). For example, a single coaxial illumination source may be mounted to each submodule of an optical module such that the illumination light produced by the coaxial illumination sources is coaxially aligned with one or more optical axes of the lens barrels in each submodule. Accordingly, in certain examples, two, three, four, or more optical and/or illumination axes of the optical head may be coaxially aligned.

In certain embodiments, each submodule and thus, each stereo channel, of an optical head may be built up and separately aligned with each other (and/or other components of the optical head) during assembly of the optical head. The individual assembly and alignment of each stereo channel facilitates easier alignment and assembly of the overall optical head, and alleviates some of the overall tolerance issues associated with assembly and alignment of multiple stereo channels together. In certain embodiments, the individual assembly and alignment of a submodule or stereo channel includes the assembly and alignment of one or more lens barrels along with one or more coaxial illumination sources. Thus, in such embodiments, lens barrels for each submodule or stereo channel may be assembled and aligned with coaxial illumination sources prior to being assembled and aligned with another submodule or stereo channel of an optical head, enabling more efficient assembly of the overall optical head.

In certain embodiments, each lens barrel of an optical module comprises a fixed focal length architecture and is configured to work with digital zoom, thereby yielding high resolution images without the need for moving components (e.g., moving lenses). In such embodiments, one lens barrel in each stereo channel may comprise a narrow field lens barrel, and the other lens barrel in each stereo channel may comprise a wide field lens barrel. In certain other embodiments, each lens barrel of the optical module comprises one or more adjustable, or moving, lenses to provide optical zoom functionality.

In certain embodiments, the two lens barrels in each stereo channel are disposed in a side-by-side arrangement. In certain other embodiments, the two lens barrels in each stereo channel are disposed in a stacked arrangement. As such, each lens barrel may “look” or receive input from the same optical axis, thereby reducing or eliminating any distortion and/or other resultant complexities, such as the need for compensation, which would arise if the lens barrels were aligned to different optical axes. In such embodiments, received light may be split between the two lens barrels in each stereo channel via a beam splitter and mirror, with the majority of light being directed into a narrow field lens barrel (e.g., the higher magnification lens barrel), while the remainder of light is directed into a wide field lens barrel. In certain embodiments, each of the lens barrels may be configured to have a relatively low f-number (f/#) and high numerical aperture (NA), thereby facilitating higher resolution and more efficient use of light to enable utilization of lower illumination levels and thus, reducing light toxicity and improving patient safety.

As a result of these and other features, the optical systems described herein provide improved overall ergonomics as compared to more conventional systems. More particularly, the optical systems described herein facilitate a low-height microscope camera that more easily enables surgeons of all physical dimensions to see over/around the optical head to display screens or other monitors placed in ergonomically advantageous positions (e.g., along a surgeon's line of sight and perpendicular to an operating room floor).

Conventional surgical systems and equipment, including visualization systems used during ophthalmic microsurgical procedures, are typically designed for users of “average” size and/or build. Thus, to use such conventional systems and equipment, surgeons having more extreme physical characteristics (e.g., height, etc.) may need to exert additional effort and/or a greater degree of strain, which can lead to chronic health issues (e.g., chronic neck and back pain) with repeated and extended use thereof. For example, a surgeon of “shorter” height may need to position themselves in non-ergonomic ways in order to see over/around a more conventional microscope camera in order to view one or more displays screens of a visualization system during, e.g., an ophthalmic surgical procedure. And, in many cases, the surgeon may need to view such display screens for extended periods of time, thus requiring the surgeon to remain in the non-ergonomic position(s) for longer and increasing the amount of physical strain on the surgeon's body. Accordingly, the optical systems described herein address this issue, and others, by enabling surgeons with more extreme physical characteristics, in addition to those with more “average” dimensions, to more comfortably view monitors and other devices in an operating room with little or no impedance from the surgical microscope camera.

Additionally, the optical systems described herein also provide improved performance compared to more conventional systems, as splitting the total zoom between multiple lens barrels offers high resolution images that enable surgeons to see ophthalmic anatomies more clearly. Even further, the optical systems described herein offer improved manufacturability, with reduced weight and manufacturing costs, as fixed focal length lens barrels, with no moving optics, are easy to fabricate, with relatively low scrap rate as well as being more robust and resistant to vibrations and misalignment. These and other benefits of the optical systems herein are elucidated in more detail below.

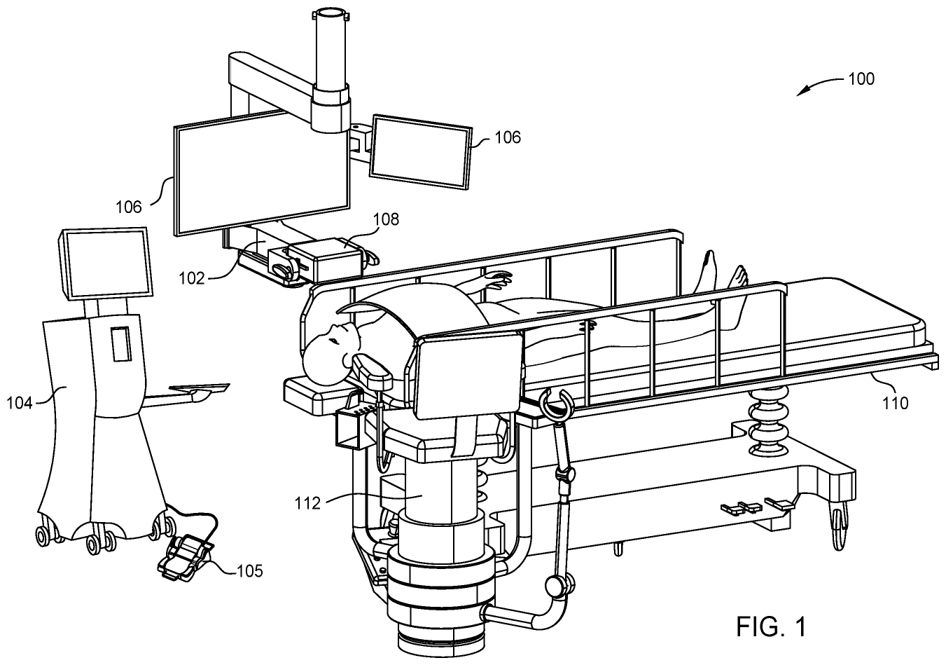

FIG. 1 illustrates a perspective view of an exemplary ophthalmic suite 100 for ophthalmic surgical procedures, according to certain embodiments of the present disclosure. Though described with reference to ophthalmic surgical procedures, those with ordinary skill in the art having the benefit of the present disclosure will readily appreciate that the disclosed technology can be applied to various other fields of anatomical diagnostics, surgical procedures, etc.

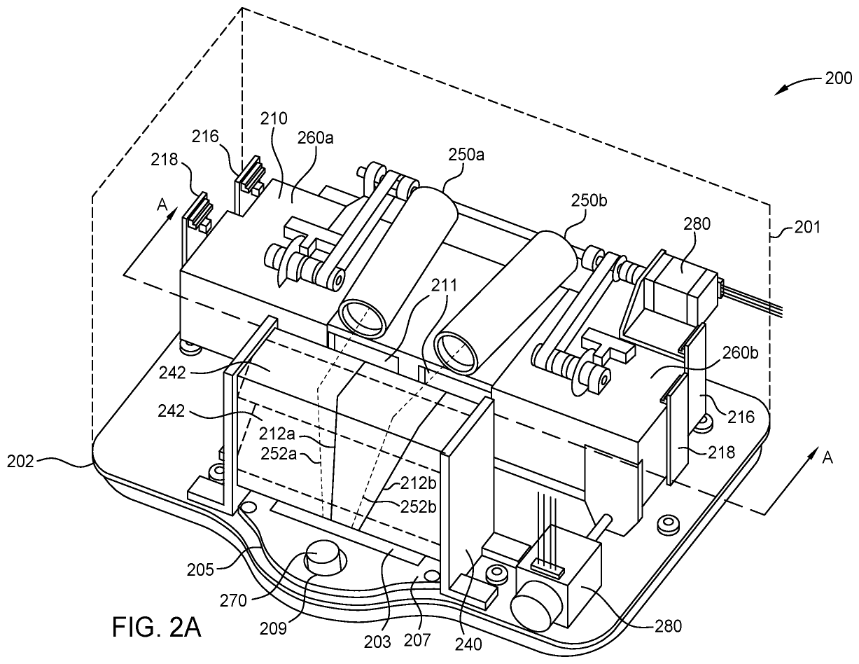

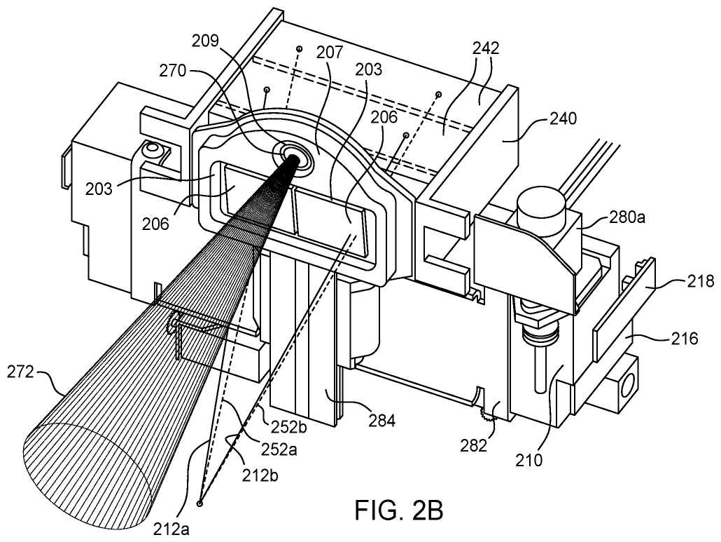

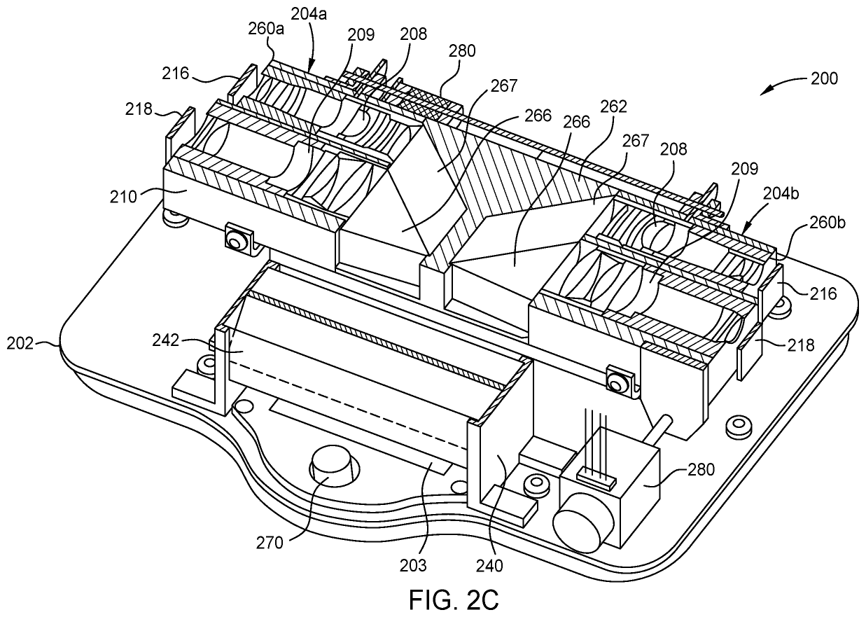

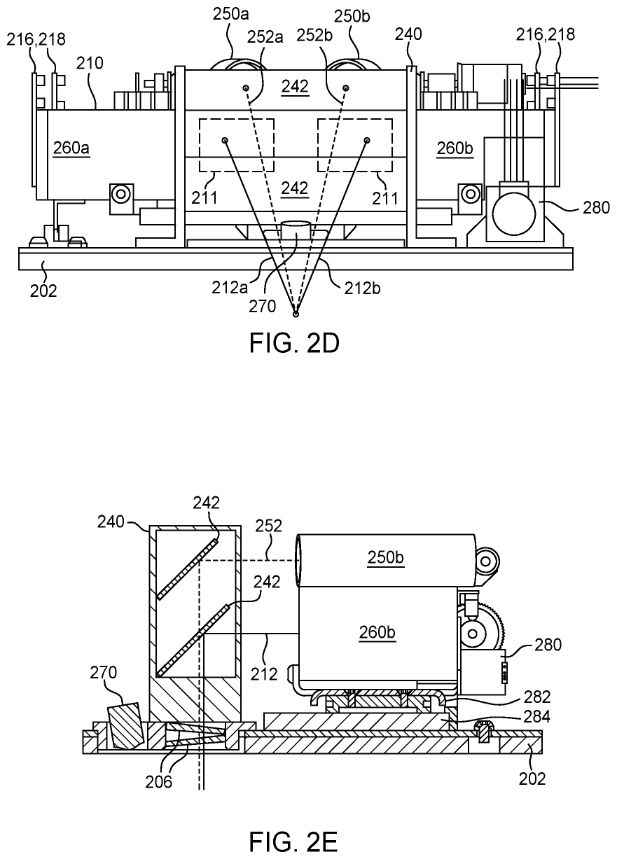

In the example of FIG. 1, ophthalmic suite 100 comprises digital visualization system 102, surgical console 104, and head-up displays 106. Digital visualization system 102 includes surgical camera 108, which is positioned over a patient's head on patient table 110. Surgical camera 108, in certain embodiments, may be a High Dynamic Range (HDR) microscope camera with resolution, image depth, clarity, and color contrast that enables high quality, three-dimensional (3D) images of patient anatomy, such as ophthalmic anatomical structures. Surgical camera 108 includes a body, or optical head, comprising a plurality of optical components (i.e., optics) for facilitating the relay and capture of 3D images of patient anatomy, which are described in further detail in FIGS. 2A-6B. Surgical camera 108 can be communicatively coupled with one or more of heads-up displays 106 (e.g., via wired connections, a wireless connection, etc.), and heads-up displays 106 can display a stereoscopic representation of a 3D image, thereby providing a surgeon, staff, or other observer(s) depth perception into a patient's eye anatomy. Surgical camera 108 can also be used to increase magnification of the eye anatomy, change a field of view, etc.

The stereoscopic representation of the 3D image can be viewed on heads-up displays 106 with stereoscopic glasses worn by the surgeon or another observer (e.g., as an autostereogram, using Fresnel lenses, etc.). With a stereoscopic representation of the 3D image displayed on heads-up displays 106, a surgeon can perform procedures on a patient's eye while being in a more ergonomic position, such as while sitting on stool 112, without bending over a microscope eyepiece and straining, e.g., their neck. Also, while conventional microscope camera designs may be tall and bulky, thus obstructing a surgeons view of, e.g., heads-up displays 106 or other equipment in the operating room, camera 108 as described herein comprises a low-profile design, thereby allowing the surgeon to see over/past camera 108 to heads-up displays 106, which may be positioned along the surgeon's line of sight.

Surgical console 104 includes a controller (not shown), and in certain embodiments, a receiver (not shown) in communication with the controller. The controller may configured to cause surgical console 104 to perform tasks associated with driving one or more devices within ophthalmic suite 100, such as digital visualization system 102 and heads-up displays 106 in certain embodiments. Accordingly, surgical console 104 can be communicatively coupled, via the receiver (e.g., via a wired connection, a wireless connection, etc.), with digital visualization system 102 (including surgical camera 108), heads-up display 106, and/or other devices within ophthalmic suite 100, such as one or more surgical probes, for performing various operations. In certain examples, surgical console 104 can receive and/or send signals to/from digital visualization system 102 for controlling parameters associated with camera 108, such as increasing/decreasing magnification, changing a field of view (FOV), applying a filter, switching between stereo channels and/or modules, etc. Surgical console 104 can also send signals to heads-up displays 106 for controlling parameters associated with image manipulation or playback, such as initiating/stopping video recording. In certain embodiments, heads-up displays 106 can also receive information, e.g., surgical parameters, from surgical console 104, and display such information on heads-up displays 106, along with the stereoscopic representation of the 3D image of a patient's eye.

Using surgical console 104, a user may control digital visualization system 102, as well as other devices within ophthalmic suite 100. In certain embodiments, the user may control such devices within ophthalmic suite 100 via adjustment of digital or physical knobs on surgical console 104, or by actuating a foot pedal 105 or other similar control device communicatively coupled to surgical console 104.

FIGS. 2A-2F illustrate various views of an exemplary optical head 200 of a surgical camera, e.g., surgical camera 108 in FIG. 1, according to certain embodiments of the present disclosure. Optical head 200 comprises an ergonomic, low-profile (e.g., low-height) design that enables surgeons of all physical dimensions to see over/around optical head 200 in order to view display screens or other monitors/equipment within an operating room setting, without additional exertion or strain.

Turning now to FIG. 2A, a simplified top perspective view of optical head 200 is illustrated, according to embodiments described herein. Optical head 200 comprises an outer casing 201, which is shown in phantom in FIG. 2A to reveal the internal components of optical head 200. Outer casing 201 encases and protects the internal components (e.g., optical module, etc., described below) of optical head 200 from damage and outside contamination, and may further provide anchoring points to stabilize such internal components within. Outer casing 201 may generally be formed of any suitable surgical-grade materials, including surgical-grade polymers, thermoplastics, thermosets, and/or elastomers. For example, outer casing 201 may comprise polycarbonate, polypropylene, polyethylene, polymethyl methacrylate, polyvinyl chloride, polyamide, acrylonitrile butadiene styrene (ABS), and the like. In certain embodiments, outer casing 201 comprises one or more surgical-grade metallic materials, including aluminum, titanium, stainless steel and other metal alloys.

Outer casing 201 couples to baseplate 202 at a bottom end thereof. Baseplate 202 serves as a base or support for internal components of optical head 200 (as well as outer casing 201), which may be rigidly or movably anchored thereto. Additionally, baseplate 202 may comprise opening 205 within which insert 207 may be removably disposed. Insert 207 may comprise window 203, which facilitates ingress and egress of light vertically in/out of optical head 200. Similar to outer casing 201, baseplate 202 may be formed of any suitable and rigid surgical-grade materials, including surgical-grade metallic materials such as aluminum, titanium, stainless steel, and other alloys. In certain embodiments, baseplate 202 comprises surgical-grade polymers, thermoplastics, thermosets, and/or elastomers, such as those described above. Together, baseplate 202 and outer casing 201 form a low-profile shape/morphology for optical head 200 in order to reduce or eliminate the potential of visual obstruction of a surgeon's line-of-sight to display screens and/or other equipment in an operating room during a surgical procedure. In the example of FIG. 2A, baseplate 202 and outer casing 201 form a generally cuboid shape, though other shapes/morphologies are also contemplated.

Within outer casing 201 and anchored to baseplate 202 is optical module 210, which internally comprises a plurality of optics (e.g., optical devices; not shown) for relaying and modulating input light (e.g., light reflected back from a patient's eye) within optical module 210 toward one or more optical sensors 216 and 218. Optical sensors 216 and 218 then receive/capture the relayed input light (as images) for display as a three-dimensional (3D) representation on a heads-up display, e.g., heads-up display 106. The optics within optical module 210 form at least two separate and independent stereo channels (e.g., a “left” channel for the user's left eye and a “right” channel for the user's right eye; not shown) to facilitate representation of relayed and captured images in 3D.

In the example of FIG. 2A, optical head 200 comprises a Greenough-style optical head, thus having optics forming two separate and independent optical axes 212a and 212b (together referred to hereinafter as optical axes 212), each for a respective “left” or “right” stereo channel. While many conventional microscope cameras utilize a common main objective (CMO)-style optical head, such style often results in tall and bulky optical heads, which can obstruct a surgeon's line-of-sight to a heads-up display during, e.g., ophthalmic procedures. Further, CMO-style optical heads typically require more lenses, thus adding to the cost and complexity of the overall surgical camera. Accordingly, utilizing a Greenough-style optical head 200 for, e.g., digital visualization system 102 enables a less obtrusive and more efficient design for surgical camera 108.

In certain embodiments, as described above, the optics for each stereo channel of optical head 200 may be comprised on a separate submodule 260, or portion, of optical module 210. For example, as shown in FIG. 2A, optical head 210 comprises two submodules 260a and 260b (e.g., “left” and “right” submodules; together referred to hereinafter as submodules 260), wherein each submodule 260 comprises optics for a different stereo channel. Accordingly, the optics of each submodule 260a and 260b form one of optical axes 212a or 212b, respectively.

As further shown in FIG. 2A, optical head 200 also includes a first mirror-beam splitter module 240 within outer casing 201 and anchored to baseplate 202. First mirror-beam splitter module 240 facilitates incoupling of input light (e.g., light reflected back from a patient's eye) into optical module 210, as well as outcoupling of any illumination light generated by light sources within optical head 200 toward a surgical site. First mirror-beam splitter module 240 comprises one or more mirrors and/or beam splitters 242, in any suitable arrangement, for redirecting input light that vertically enters optical head 200 through window 203 of insert 207 into optical module 210. Accordingly, first mirror-beam splitter module 240 may be positioned over/adjacent to a window 203, which functions as an aperture for optical head 200. Similarly, optical module 210 may comprise one or more window(s) 211, which enable the input light to pass to one or more optics of the optical module 210 for further relay and/or manipulation. In certain embodiments, first mirror-beam splitter module 240 is configured to horizontally transmit, e.g., redirect, input light into optical module 210 at an angle between about 45° and about 135° relative to an initial orientation of the input light.

Optical head 200 may further include one or more coaxial illumination sources 250 configured to generate and transmit illumination light for illuminating a surgical site, e.g., the inner structures of a patient's eye, during a surgical procedure. In the example of FIG. 2A, optical head 200 comprises two coaxial illumination sources 250a and 250b (together referred to hereinafter as coaxial illumination sources 250), each generating and transmitting illumination light along an illumination axis 252a or 252b (together referred to hereinafter as illumination axes 252), respectively. Each illumination axis 252 is coaxially aligned with an optical axis of the optical module 210—here, illumination axis 252a is aligned with optical axis 212a, and illumination axis 252b is aligned with optical axis 212b. Alignment of the illumination axes 252 and optical axes 212 facilitates optimal illumination, and thus, brightness, of images captured by optical head 200.

In certain embodiments, coaxial illumination sources 250 may be mounted or assembled within optical head 200 in any suitable arrangement to facilitate coaxial alignment of illumination axes 252 and optical axes 212. For example, coaxial illumination sources 250 may be mounted directly to optical module 210. In some examples, each coaxial illumination source 250 may be mounted to a submodule 260 of optical module 210. During assembly of optical head 200, each optical axes 212 may be aligned to an illumination axis 252 separately, prior to the optical axes 212 being aligned together. Accordingly, upon aligning each optical axis 212 with a respective illumination axis 252, the illumination axes 252 and optical axes 212 may stay aligned during further downstream assembly of optical head 200.

In certain embodiments, coaxial illumination sources 250 may be disposed outside of optical head 200, and light generated thereby may be transmitted into optical head 200, along illumination axes 252, via a liquid light pipe, fiber optic cable, and the like.

Generally, any suitable types of illumination light sources may be utilized for coaxial illumination sources 250. For example, in certain embodiments, coaxial illumination sources 250 comprise light-emitting diodes (LED), such as white light LEDs, red-green-blue (RGB) light LEDs, and the like. In certain embodiments, coaxial illumination sources 250 comprise incandescent lamps, such as halogen or tungsten-halogen lamps.

In addition to coaxial illumination sources 250, optical head 200 may also include one or more oblique illumination sources 270. In the example of FIG. 2A, a single oblique illumination source 270 is shown disposed through opening 209 of insert 207, which may be adjacent to window 203. Oblique illumination source(s) 270 may generate and transmit additional illumination light 272 (shown in FIG. 2B) for increased illumination of a surgical site during a surgical procedure. In certain embodiments, oblique illumination source(s) 270 may be arranged/configured to generate and transmit illumination light 272 along one or more illumination axes (not shown) disposed at an angle relative to optical axes 212 of optical module 210, thus not being coaxially aligned therewith. Accordingly, this “oblique” angle of illumination light 272 generated by oblique illumination source(s) 270 facilitates illumination of tissues peripheral to a surgical site in addition to the surgical site itself. Generally, oblique illumination source(s) 270 may comprise the same type, or different type, of illumination light source as coaxial illumination sources 250.

Further shown in FIG. 2A are actuators 280 and their associated components (e.g., gears, rods, belts, etc.). Actuators 280 may be utilized to actuate various components of optical head 200, such as optical module 210, as well as one or more filters or apertures of optical module 210, as described in more detail below. Actuators 280 may comprise any suitable controlled motion motors, such as servo motors. In certain embodiments, actuators 280 comprise stepper motors, such as permanent magnet (PM) stepper motors, variable reluctance (VR) stepper motors, and hybrid synchronous (HY) stepper motors. In such embodiments, the stepper motors may be controlled via full-step positioning, rather than micro-step positioning, in order to generate less heat. Micro-stepping the motors may require considerable current to be sent to the motors to maintain a micro-step position (generating considerable heat), while full-stepping the motors requires just enough current to keep the coils thereof activated (generating less heat). The stepper motors may thus be micro-stepped for smoother quieter motions while running, but may be stopped at full steps to reduce heat generation. Reduced heat generation facilitates reduced movement of optics within optical head 200 during use, and thus, the alignment of such optics may be better maintained. Generally, actuators 280 described herein may be controlled by a user, e.g., a surgeon or other medical practitioner, via any suitable methods or mechanisms. For example, in certain embodiments, actuators 280 may be controlled via a foot switch, voice commands, surgical console, and/or other mechanisms used to control/toggle devices, functions, or parameters of surgical systems.

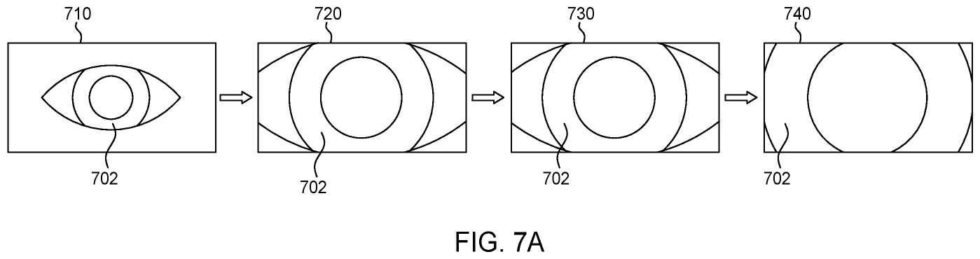

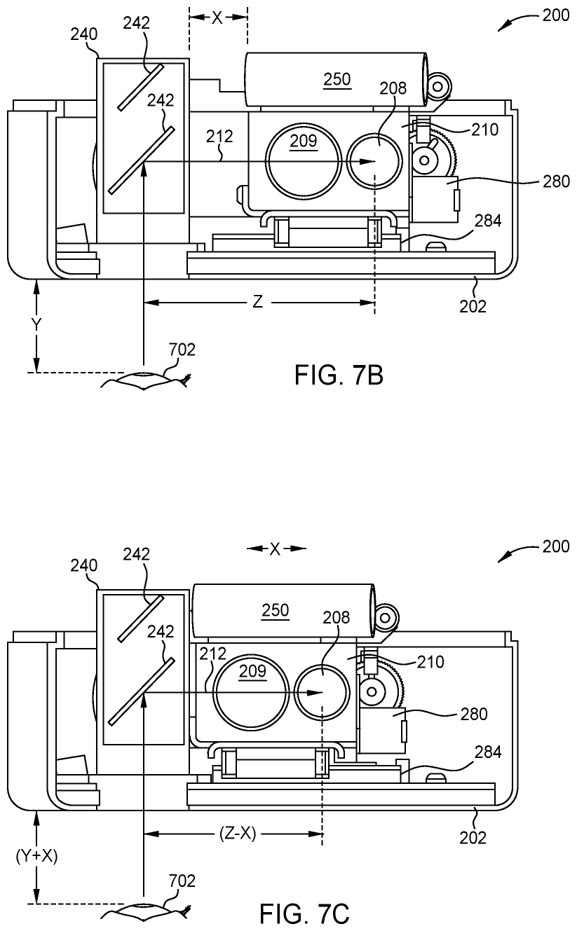

FIG. 2B illustrates a bottom perspective view of the internal components of the optical head in FIG. 2A, according to certain embodiments of the present disclosure. In FIG. 2B, baseplate 202 is removed for clarity. As shown, optical module 210 is movably coupled to rail 284, which may be anchored to baseplate 202 of optical head 200. Sliding of optical module 210 along rail 284 facilitates translational movement of optical module 210 toward and/or away from first mirror-beam splitter module 240 in optical head 200. This movement of optical module 210 relative to first mirror-beam splitter module 240 enables adjustment of the working distance of optical head 200 during a surgical procedure, as well as focusing of images captured by optical module 210, which is described in more detail below with reference to FIGS. 7B and 7C. In certain embodiments, optical module 210 is coupled directly to rail 284. In other embodiments, optical module 210 is indirectly coupled to rail 284 via platform 282, which is movably (e.g., slidably) coupled to rail 284 and to which optical module 210 may be anchored via, e.g., bolting.

Translation of optical module 210 along rail 284 is driven by at least one of actuators 280. In the example of FIG. 2B, actuator 280a is shown, which may comprise a stepper motor for driving the movement of optical module 210 along rail 284, thereby adjusting the working distance and/or focusing of optical head 200. Actuator 280a, like other actuators 280, may be controlled via user input. For example, in certain embodiments, a surgeon or other medical practitioner may control actuator 280a via a foot switch, voice commands, surgical console, and/or other suitable methods and mechanisms used to control/toggle devices, functions, or parameters of surgical systems.

FIG. 2B further depicts illumination light 272 as generated by an oblique illumination source 270. Oblique illumination source 270 may be disposed at least partially through, or adjacent to, opening 209 of insert 207, which enables propagation of illumination light 272 from oblique illumination source 270 toward a surgical site (and out of optical head 200). Insert 207, which may be removably attached to baseplate 202, further comprises window 203 for facilitating transmission of light in/out of optical module 210. Thus, insert 207, and more particularly, window 203, are disposed below first mirror-beam splitter module 240, which splits and/or redirects light between optical module 210 and window 203. In certain embodiments, window 203 comprises wedge optics 206 to reduce the creation of optical ghosts and other artifacts in images produced by optical head 200. Window 203 may generally comprise one or more of such wedge optics 206 (e.g., in a stacked arrangement) per optical axis 212 of optical module 210 that passes through window 203. In the example of FIG. 2B, at least two wedge optics 206 are shown (one for each optical axis 212a and 212b) arranged side-by-side. In certain embodiments, window 203 may further comprise a stationary filter, such as an infrared (IR) or laser light filter. For example, in certain embodiments, the stationary filter may comprise a coating on an optic of window 203.

Note that although opening 209 and window 203 are depicted as features of insert 207 in the examples of FIGS. 2A and 2B, opening 209 and window 203 may be formed directly in baseplate 202 in other examples (instead of opening 205). However, utilization of insert 207 may facilitate easier access for repair and/or replacement of various components of optical head 200, including oblique illumination source 270 and/or wedge optics 206.

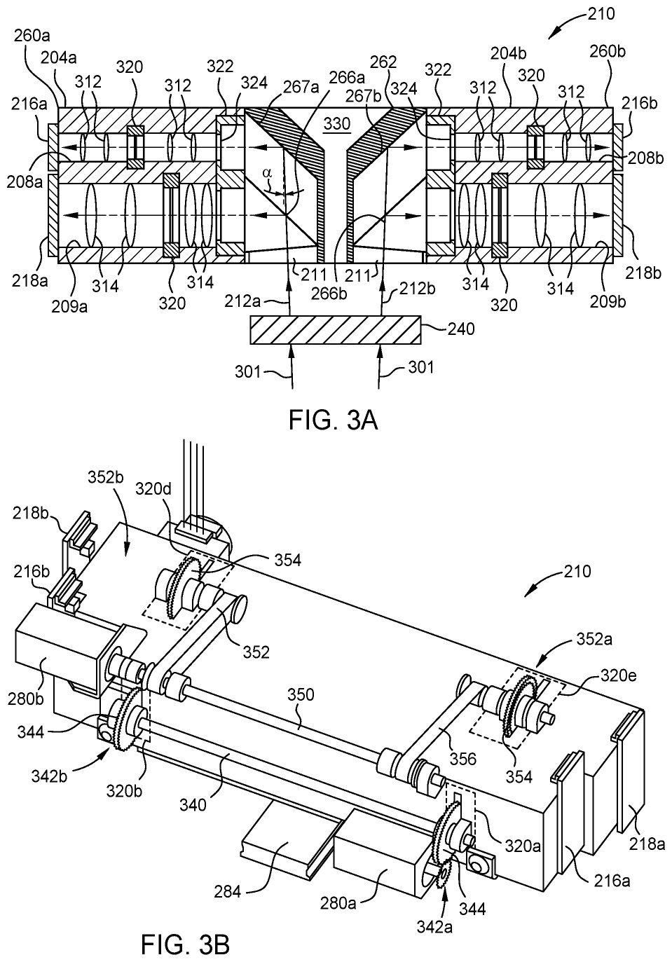

FIG. 2C illustrates another top perspective view of optical head 200, wherein optical module 210 of optical head 200 is shown in cross-section along the plane “A-A” in FIG. 2A to reveal the optics therein, according to certain embodiments of the present disclosure. As shown, optical module 210 comprises two separate and independent channels 204a and 204b (e.g., one for each user's eye; together referred to hereinafter as channels 204), which may each be disposed within a separate and independent submodule 260a or 260b of optical module 210. Within each channel 204 are a plurality of lens barrels. In the example of FIG. 2C, two lens barrels 208 and 209 are shown in each channel 204, which work in tandem to break up the optical workload for each corresponding channel 204, described in more detail with reference to FIG. 3A below. Each lens barrel 208 or 209 respectively comprises a plurality of optics, such as lenses, for relaying and/or modulating input light that has entered optical module 209, e.g., along optical axes 212a or 212b, to optical sensors 216 and 218. Input light is redirected into each lens barrel 208 or 209 via one or more second mirror-beam splitter modules 262 disposed within optical module 210 and comprising beam splitters 266 and mirrors 267. In certain embodiments where optical module 210 comprises submodules 260a and 260b, the one or more second mirror-beam splitter modules 262 may be disposed between submodules 260a and 260b within optical module 210. In certain embodiments, second mirror-beam splitter module(s) 260 are configured to horizontal transmit, e.g., redirect, input light into lens barrels 208 or 209 at an angle between about 45° and about 135° relative to an orientation of the input light as transmitted by first mirror-beam splitter module 240.

FIGS. 2D and 2E illustrate the coincident axes of optical module 210 and coaxial illumination sources 250, according to certain embodiments of the present disclosure. More particularly, FIG. 2D depicts the coincident arrangement optical axes 212 and illumination axes 252 in a front view of the of the internal components of optical head 200, while FIG. 2E illustrates these coincident axes in a side view of optical head 200. As shown, optical axis 212a of optical module 210, formed by one of the two channels 204 thereof, is coincident with illumination axis 252a of coaxial illumination source 250a. Meanwhile, optical axis 212b of optical module 210, formed by the other one of two channels 204 thereof, is coincident with illumination axis 252b of coaxial illumination source 250b. And, each pair of coincident axes 212, 252 converge with one another to form a focal point, as shown in FIG. 2D. The coincident nature of the optical axes 212 and respective illumination axes 252 is facilitated by the relative orientation of channels 204 (e.g., submodules 260) of optical module 210 and coaxial illumination sources 250, as well as the arrangement and orientation of first mirror-beam splitter module 240 and second mirror-beam splitter module(s) 262, which converge the axes 212 onto one another. Note that in FIGS. 2D and 2E, coincident optical axes 212 and illumination axes 252 are depicted adjacent to one another for clarity.

FIG. 3A illustrates a simplified cross-sectional side view optical module 210 with optical axes 212 thereof, according to certain embodiments of the present disclosure. As shown, input light 301 (e.g., light reflected back from a patient's eye) is transmitted toward optical module 210 along optical axes 212a and 212b (together, optical axes 212) of two stereo channels 204a and 204b (together, channels 204), respectively, via first mirror-beam splitter module 240. Again, each channel 204a and 204b may be comprised in a separate submodule 260a or 260b of optical module 210, which are assembled and aligned with one another. The input light 301 is then optionally split and relayed by optics disposed adjacent or within the plurality of lens barrels (e.g., two or more lens barrels) of each channel 204 before reaching a plurality of optical sensors 216 and 218 for capture and display of the light as a 3D representation on a heads-up display, e.g., heads-up display 106.

As shown, each channel 204a and 204b comprises at least a first lens barrel 208a or 208b (together referred to hereinafter as first lens barrels 208), respectively, and a second lens barrel 209a or 209b (together referred to hereinafter as second lens barrels 209), respectively. The first lens barrel 208 and second lens barrel 209 in each channel 204 work in tandem to break the optical workload for the corresponding channel 204 into multiple optics (e.g., lens) systems for wide field versus narrow field viewing, which in certain embodiments facilitates an overall fixed focal length system for optical head 200 with no moving optics. As a result of the two barrels splitting the optical workload, a user, e.g., a surgeon, can instantly cycle between the first lens barrel 208 and the second lens barrel 209 in each channel 204 to obtain the different fields of view (e.g., wide versus narrow framings). Additionally, with no moving lenses, optical module 210 enables an “instant” zoom, requiring only milliseconds or less to zoom from one magnification to another via digital mechanisms. Further, utilizing two barrels in tandem for each channel 204 enables a picture-in-picture view, wherein two different views may be observed by the user at once. And, by splitting the total number of optics for each channel between two barrels, tolerances for the optics within the individual barrels may be more lax (since all the optics do not have to be arranged within the same barrel of the channel), thus making fabrication of optical head 200 easier.

To facilitate splitting of the optical workload between lens barrels, input light 301 may be split and reflected by one or more beam splitters 266a or 266b (together referred to hereinafter as beam splitters 266) and one or more mirrors 267a or 267b (together referred to hereinafter as mirrors 267) of second mirror-beam splitter module(s) 262. In certain embodiments, beam splitters 266 and/or mirrors 267 may comprise optical prisms with one or more reflective or partially reflective surfaces, or other suitable optic devices.

In the example of FIG. 3A, a single second mirror-beam splitter module 262 is disposed between submodules 260a and 260b of optical module 210. The single second mirror-beam splitter module 262 comprises two sets of beam splitters 266a and 266b, as well as two sets of mirrors 267a and 267b, for directing input light 301 (transmitted along optical axes 212a and 212b) in opposing directions through lens barrel tandems 208a and 209a, or 208b and 209b. In certain other embodiments, two or more second mirror-beam splitter modules 262 may be utilized, wherein each second mirror-beam splitter module 262 comprises a single set of beam splitters 266 and/or mirrors 267. In any configuration, however, the arrangement of second mirror-beam splitter module(s) 262 facilitates the coaxial alignment of the optical axes of multiple lens barrels in each stereo channel 204 to form an optical axis 212 of optical module 210. And, when utilized with a coaxial illumination source 250, three or more optical axes (e.g., the optical axes of two lens barrels 208, 209 in a stereo channel 204, plus the optical axis of a coaxial illumination source 250) may be aligned (i.e., coincident) by optical head 200. Accordingly, the angles beam splitters 266 and/or mirrors 267 of second mirror-beam splitter module(s) 262 define the optical axis angle α of the Greenough-style system of optical head 200. In certain embodiments, the angle α is between about 1° and about 20°, such as between about 1° and about 15°, such as between about 5° and about 10°. In certain embodiments, the angle α is about 5°.

In certain embodiments, first lens barrels 208 include wide field lens barrels each having a plurality of lenses and/or other optics 312 configured to create wide field images of patient anatomy from input light 301 and project the images onto optical sensors 216a or 216b (together referred to hereinafter as optical sensors 216). In certain embodiments, second lens barrels 209 include narrow field lens barrels each having a plurality of lenses and/or other optics 314 configured to produce magnified, narrow field images of the patient anatomy and project the images onto optical sensors 218a or 218b (together referred to hereinafter as optical sensors 218). During use, a surgeon can instantly switch between the wide field and narrow field lens barrels (e.g., switch between images captures by optical sensors 216 and 218), which are configured to provide a seamless transition between wide field and narrow field views of the patient anatomy. In certain embodiments, optics 312 of the wide field first lens barrels 208 may comprise Risley prisms, or other wedge-type optics, disposed at ends of first lens barrels 208 nearest second mirror-beam splitter module(s) 262 (e.g., opposite sensors 218), which may be distinct from other optics within lens barrels 209. Such Risely prisms enable the alignment of the optical axes of wide field first lens barrels 208 with those of narrow field lens barrels 209. In such embodiments, the Risley prisms or other wedge-type optics are rotationally adjustable for “steering” of the optical path therethrough (into lens barrels 209).

Optical sensors 216 and 218 may comprise any suitable types of imaging sensors. In certain embodiments, optical sensors 216 and 218 comprise ultra high definition sensors with a resolution of 4K or more. For example, in certain embodiments, optical sensors 216 and 218 comprise ultra high definition sensors with a resolution of 8K or more. In certain embodiments, optical sensors 216 and 218 comprises sensors that are rated at a resolution that is twice or more than that of a displayed (e.g., output) resolution. Utilizing sensors rated at twice the output resolution facilitates maintenance of the very high resolution provided by the fixed focal length system of optical module 210 at all zoom levels up to 2× magnification or 4× magnification.

As shown in FIG. 3A, in certain embodiments, the barrels in channels 204 of optical module 210 may be arranged in an opposing layout; that is, first lens barrel 208a of channel 204a may be disposed opposite of and in reverse (but parallel) orientation to first lens barrel 208b of channel 204b, and second lens barrel 209a of channel 204a may be disposed opposite of and in reverse orientation to second lens barrel 209b of channel 204b. Such an opposing layout of the lens barrels enables a compact, robust, and manufacturing-friendly design. For example, the opposing layout of optical module 210 facilitates improved thermal performance of optical head 200 and thus, surgical camera 108, as the opposing lens barrels will move in tandem, thus maintaining their coaxial arrangement, during states of thermal variation. With more conventional microscope camera designs, such as CMO-style microscope cameras, optics are typically arranged side-by-side. As these cameras heat up during use, the heat may cause the optics to move around and shift, typically away from each other, thereby creating misalignment of the optics and distorting any 3D images collected therefrom. However, with the opposing layout of optical module 210, optic misalignment due to thermal load may be reduced and/or eliminated.

In certain embodiments, first lens barrels 208 and second lens barrels 209 are designed to have a low f-number (f/#) to facilitate higher resolution and more efficient use of light. The more efficient use of light by the lens barrels enables utilization of lower illumination levels of a patient's eye, thereby reducing light toxicity to the patient's eye. In certain embodiments, first lens barrels 208 and second lens barrels 209 may have an f-number of about f/8, f/6, f/5, f/4, f/3, f/2, or the like. In certain embodiments, first lens barrels 208 and second lens barrels 209 have an f-number between about f/8 and about f/4. In some examples, an f-number lower than f/4 may result in optics that are large an non-ergonomic, while an f-number greater than f/8 may result in a low image resolution.

As described above, to facilitate tandem operation of first lens barrels 208 and second lens barrels 209 in each channel 204, input light 301 is split and/or reflected by beam splitters 266 and mirrors 267 of second mirror-beam splitter module 262 into the corresponding barrel tandems in each channel. In certain embodiments, input light 301 is split about 50:50 between the first lens barrel 208 and the second lens barrel 209 in each channel; in certain embodiments, input light 301 is split about 60:40 between the first lens barrel 208 and the second lens barrel 209 in each channel 204, or vice versa; in certain embodiments, input light 301 is split about 65:35 between the first lens barrel 208 and the second lens barrel 209 in each channel 204, or vice versa; in certain embodiments, input light 301 is split about 70:30 between the first lens barrel 208 and the second lens barrel 209 in each channel 204, or vice versa; in certain embodiments, input light 301 is split about 75:25 between the first lens barrel 208 and the second lens barrel 209 in each channel 204, or vice versa; in certain embodiments, input light 301 is split about 80:20 between the first lens barrel 208 and the second lens barrel 209 in each channel 204, or vice versa; in certain embodiments, input light 301 is split about 85:15 between the first lens barrel 208 and the second lens barrel 209 in each channel 204, or vice versa; in certain embodiments, input light 301 is split about 90:10 between the first lens barrel 208 and the second lens barrel 209 in each channel 204, or vice versa; or, in certain embodiments, input light 301 is split about 95:5 between the first lens barrel 208 and the second lens barrel 209 in each channel 204, or vice versa. In certain embodiments, a majority of the input light 301 is reflected into a corresponding second lens barrel 209, e.g., the narrow field lens barrel, since the narrow field lens barrel has a higher magnification, and many, if not most, ophthalmic surgical procedures are performed while the surgeon is viewing patient anatomy under the higher magnification.

Optical module 210, and thus, digital visualization system 102, may comprise any suitable types of magnification/zoom mechanisms for magnifying images produced by first lens barrels 208 and second lens barrels 209. For example, in certain embodiments, zoom/magnification for first lens barrels 208 and second lens barrels 209 may be controlled by digital mechanisms, e.g., digital cropping and rescaling mechanisms via a software on digital visualization system 102 or surgical console 104, thus facilitating smaller columns (e.g., bodies) for each barrel and not requiring any moving parts therein (e.g., moving optics 212 or 214). In such embodiments, lens barrels 208 and 209 may comprised fixed focal length optics 312 or 314, which make fabrication of such lens barrels easier. In certain embodiments, zoom/magnification for first lens barrels 208 and second lens barrels 209 may be controlled by mechanical mechanisms (i.e., optical mechanisms), thus facilitating better image resolution but requiring moving optics 312 or 314. In still other embodiments, one of each lens barrel tandem may be configured for use with a digital zoom mechanism and the other barrel may comprise a mechanical zoom mechanism. For example, first lens barrels 208 may comprise a digital zoom mechanism, while second lens barrels 209 may include a mechanical zoom mechanism, thus providing a “hybrid” zoom system for optical module 210.

Optics 312 and/or optics 314 may generally include any suitable types of optical components for relaying input light 301 to optical sensors 216 or 218. For example, optics 312 and/or optics 314 may comprise relay lenses, condensing lenses, diffractive elements, beam splitters, and/or other types of optical relay devices. In certain embodiments, first lens barrels 208 and second lens barrels 209 may further comprise one or more irises, one or more filters, and/or other similar light modulation devices. In the example of FIG. 3A, each first lens barrel 208 and second lens barrel 209 comprises an iris 320, which facilitates control of the amount of light being passing through the respective lens barrel and changes a depth of field during viewing. Irises 320 may be opened/closed via actuation by one or more actuators 280, as described with reference to FIG. 3B below. Additionally, optical module 210 further comprises a filter block 322 for each channel 204, which includes one or more filters 324 (e.g., color filters, infrared filters, laser light filters, etc.) for each lens barrel of the channel. Similar to irises 320, filters 324 of filter blocks 322 may be selected via translation, e.g., sliding, of filter blocks 322 into and out of place, as driven by one or more actuators 280. In further embodiments, one or more stationary or movable filters may be mounted externally to optical module 210 within optical head 200, such as adjacent to window(s) 211 thereof (not shown). In specific embodiments, an external stationary filter may comprise a coating on an optic of window 203, such as an IR filter coating on a proximal or distal surface of an optic of window 203. In specific embodiments, an external stationary filter may comprise a laser filter mounted to insert 207 and with dimensions defined by window 203.

In certain embodiments, optical module 210 further comprises conduit 330. Conduit 330 may comprise an opening, channel, or duct formed through optical module 210 in which an OCT fiber optic probe, a laser range-finding probe, an oblique illumination source, a fixation light source, or the like may be disposed for use during a surgical procedure. Conduit 330 may generally be centrally disposed in optical module 210, e.g., between channels 204. For example, in FIG. 3A, central channel 330 is disposed through second mirror-beam splitter module 262 between the sets of beamsplitters 266 and mirrors 267 thereof. In examples where two (or more) second mirror-beam splitter modules 262 are utilized, however, central channel 330 may be disposed between such modules.

FIG. 3B illustrates a rear perspective view of optical module 210, along with actuators 280 coupled thereto, according to certain embodiments of the present disclosure. As described above, optical module 210 may comprise one or more movable irises 320, as well as one or more movable filters 324, which can each be driven/actuated by one or more actuators 280. In certain embodiments, a single actuator 280 may drive a single iris 320, and/or a single filter 324. In certain embodiments, a single actuator 280 may drive two or more irises 320, and/or two or more filters 324 for efficiency and synchronization of lens barrels in different channels 204 in optical module 210. For example, in FIG. 3B, two motors 280a and 280b are depicted at the rear of optical module 210 for driving four irises 320a-d (shown in phantom). Motor 280a is indirectly coupled to one iris 320a or 320b in each of lens barrels 208a and 208b (shown in FIG. 3A) via axle 340 and a set of direct- or belt-drive assemblies 342a and 342b (shown as direct-drive assemblies in FIG. 3B). Each drive assembly 342 may comprise one or more gears 344 and/or belts (if belt-driven) coupling axle 340 to iris 320a or 320b for delivering power thereto. Similarly, motor 280b is indirectly coupled to one iris 320c or 320d in each of lens barrels 209a and 209b via axle 350 and a set of belt- or direct-drive assemblies 352a and 352b (shown as belt-drive assemblies in FIG. 3B), wherein each drive assembly 352 may comprise one or more gears 354 and/or belts 356 coupling axle 350 to irises 320c, 320d. Accordingly, one motor, 280a, may drive both “left” stereo channel and “right” stereo channel irises 320a, 320b in lens barrels 208 (e.g., the wide field irises), while another motor, 280b, may drive both “left” stereo channel and “right” stereo channel irises 320c, 230d in lens barrels 209 (e.g., the narrow field irises).

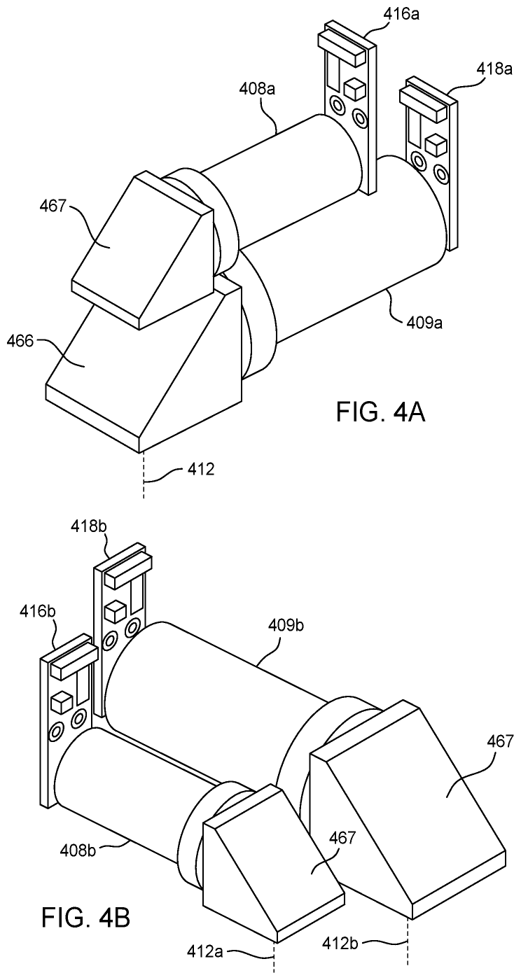

FIG. 4A illustrates a perspective side view of an exemplary tandem of lens barrels of optical module 210 in a first arrangement, according to certain embodiments of the present disclosure. In the example of FIG. 4A, first lens barrel 408a and second lens barrel 409a, which may be representative of a tandem of barrels 208 and 209 in either channel 204 of FIG. 3A, are stacked upon each other. Here, first lens barrel 408a, which comprises a wide field lens barrel, is stacked over second lens barrel 409a, which comprise a narrow field lens barrel. In such an arrangement, first lens barrel 408a and second lens barrel 409a are disposed along and receive input light from the same optical axis 412 of optical module 210 and thus, optical head 200. Input light passing along optical path 412 must therefore be split and/or reflected by one or more beam splitters 466 and mirrors 467 to facilitate light being directed into each barrel and onto optical sensors 416a, 418a thereafter. Though less light may be directed into each barrel as a result, arranging the tandem of barrels in a stacked arrangement facilitates no loss or change of perspective when switching between views from first lens barrel 408a and second lens barrel 409a.

FIG. 4B illustrates a perspective side view of another exemplary tandem of lens barrels of optical module 210 in a second arrangement, according to certain embodiments of the present disclosure. In the example of FIG. 4B, first lens barrel 408b and second lens barrel 409b, which may be representative of a tandem of barrels 208 and 209 in either channel 204 of FIG. 3A, are arranged side-by-side. In such an arrangement, first lens barrel 408b and second lens barrel 409b are each disposed along and receive input light from a separate optical axis 412a or 412b of optical head 200, and thus no light splitting is required to direct input light into each barrel and thereafter onto optical sensors 416b, 418b. Instead, mirrors 467 may be utilized to simply reflect input light into each barrel, separately. However, because the barrels are disposed along different optical paths, switching views between first lens barrel 408b and second lens barrel 409b creates a loss or change of perspective for the user observing the heads-up display.

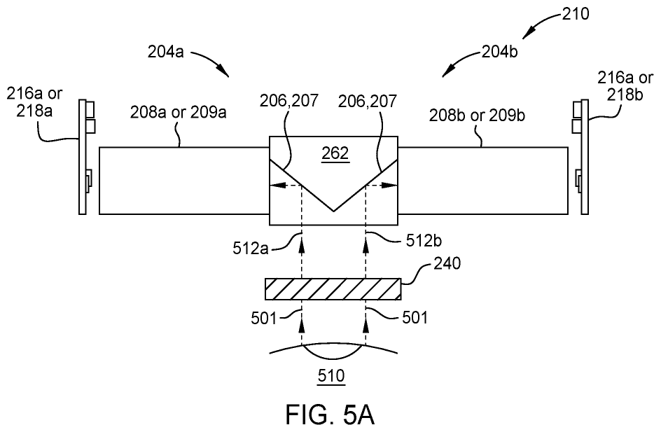

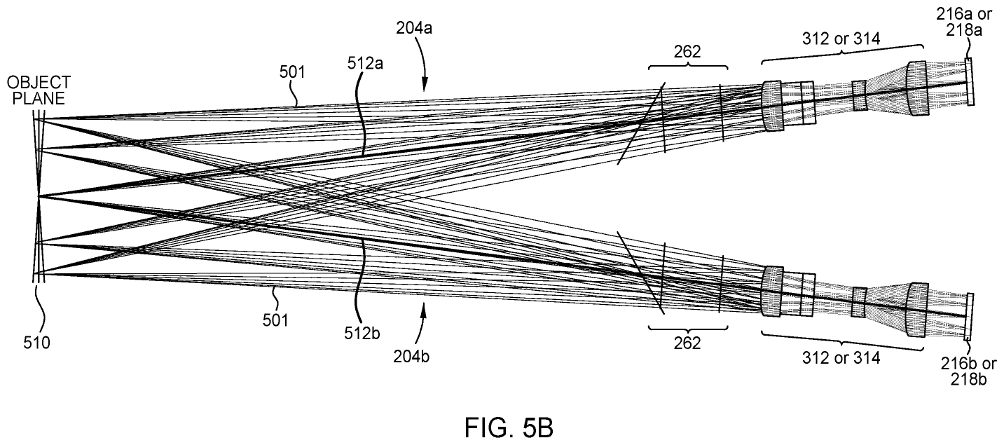

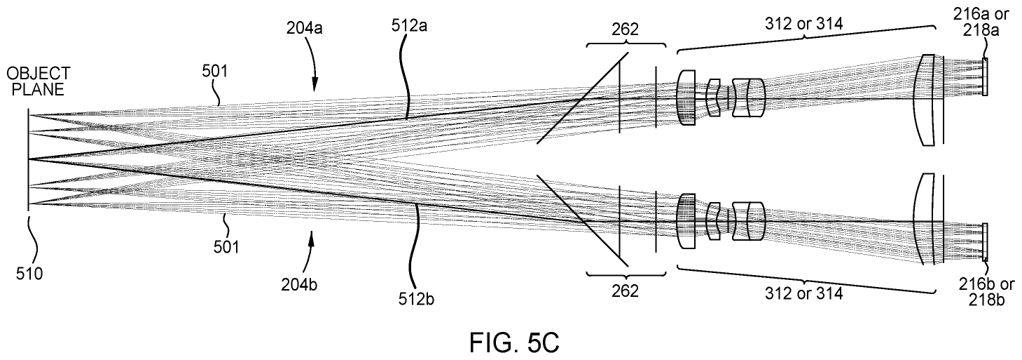

FIG. 5A illustrates a schematic cross-sectional side view of optical head 200, including optical module 210, while FIGS. 5B and 5C schematically illustrate different exemplary orientations of the optics in lens barrels 208 and 209 of optical module 210, according to certain embodiments of the present disclosure. FIGS. 5A-5C are herein described together for clarity. As shown in FIG. 5A and noted above, input light 501 is reflected from a patient's eye 510 into optical module 210 along optical axes 512a and 512b by first mirror-beam splitter module 240. Within optical module 210, the input light 501 is then directed into barrels 208a, 208b and/or 209a, 208b by second mirror-beam splitter module(s) 262. Accordingly, the angle of mirrors 267 and/or beam splitters 266 of second mirror-beam splitter module(s) 262, in addition to the angle of optics 312 or 314 (shown in FIG. 3A) within lens barrels 208 or 209, respectively, may result in the lens barrels having either an emulated “tilted” orientation, as shown in FIG. 5B, or an emulated “straight” orientation, as shown in FIG. 5C.

In the “tilted” orientation of FIG. 5B, the major axes of lens barrels 208 and 209, and thus, the major axes of optics 312 and 314, are disposed at a zero degree angle with respect to optical axes 512 of input light 501, as transmitted into the lens barrels 208 and/or 209 by second mirror-beam splitter module(s) 262. This emulates a more conventional microscope arrangement wherein the optics for different stereo channels are disposed at non-zero angles relative to one another (e.g., the stereo channels are “tilted”). Such an orientation enables sensors 216 and 218 to be centrally aligned (e.g., “in-line”) with the major axes of their respective lens barrels 208 and 209, and further enables the utilization of smaller-dimension optics 312 and 314. In certain embodiments, the tilted configuration may cause an optical distortion known as the “keystone effect” in images collected by optical sensors 216 and 218; however, this distortion can be compensated for by software-based distortion correction.

In the straight orientation of FIG. 5C, the major axes of lens barrels 208 and 209, and thus, the major axes of optics 312 and 314, are disposed at a non-zero degree angle with respect to optical axes 512 of input light 501, as transmitted into the lens barrels 208 and/or 209 by second mirror-beam splitter module(s) 262. This emulates a more conventional microscope arrangement wherein the optics for different stereo channels are disposed parallel relative to one another (e.g., the stereo channels are “straight”). Unlike the tilted configuration, the straight configuration may not require compensation or correction for keystone-like distortion, but may be more complex to assemble and may require larger dimension optics and offset sensors 216 and 218 (e.g., unaligned with the major axes of their respective lens barrels 208 and 209).

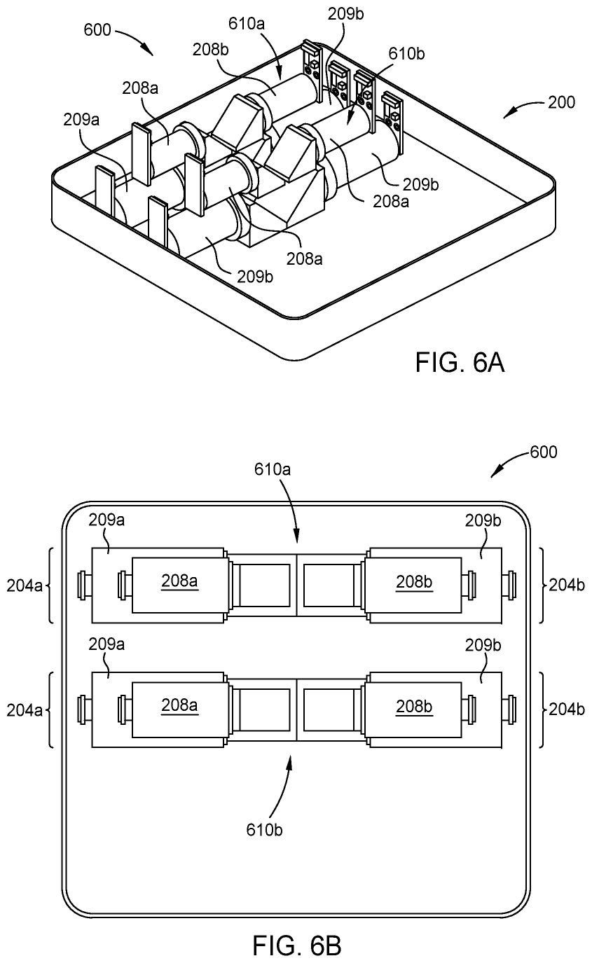

FIG. 6A schematically illustrates a perspective view of another exemplary configuration 600 of optical head 200 for use with multiple different types of ophthalmic procedures, according to certain embodiments of the present disclosure. FIG. 6B illustrates a plan view of exemplary configuration 600 of FIG. 6A, according to certain embodiments of the present disclosure. Thus, FIG. 6A and FIG. 6B are herein described together for clarity.

Generally, optical head 200 may be configured for use with multiple different types of surgical procedures, such as cataract procedures, retinal procedures, and other types of ophthalmic surgical procedures. In certain embodiments, optical head 200 may comprise multiple groups of lens barrels in an optical module, or multiple optical modules, wherein each group or optical module is optimized for performing one or more different types of surgical procedures. In the exemplary configuration 600 shown in FIGS. 6A and 6B, optical head 200 comprises two optical modules 610a and 610b (together referred to hereinafter as modules 610), wherein each module 610 is optimized for performing at least one different type of surgical procedure. In FIGS. 6A and 6B, only the lens barrels of optical modules 610 are shown for clarity. As depicted, each module 610 comprises two channels 204a and 204b, one for each eye, wherein each channel 204 comprises a first lens barrel 208a or 208b, and a second lens barrel 209a or 209b, working in tandem as described above. Configuration 600 of optical head 200 thus comprises a total of eight lens barrels, though additional optical modules 610 and thus, additional lens barrels, may be utilized in other embodiments.

Optical modules 610 may be arranged in any suitable arrangement and oriented in any suitable orientation in relation to each other. In the example of FIG. 6B, optical modules 610a and 610b are shown as being disposed in a side-by-side arrangement and parallel orientation, though other arrangements and/or orientations are further contemplated. Prior to or during a surgical procedure, a user, e.g., a surgeon, can select or switch between each opical module 610 by selecting or adjusting a digital or physical knob on a surgical console, e.g., surgical console 104, a digital or physical knob on a digital visualization system, e.g., digital visualization system 100, or by actuating a foot pedal or similar mechanism. In response, a controller in communication with the surgical console and/or digital visualization system may switch views between optical modules 610.

As described above, each optical module 610 in FIGS. 6A and 6B may be optimized for performance of a different ophthalmic surgical procedure, e.g., a cataract/anterior procedure or a retinal/posterior procedure. Accordingly, in certain embodiments, each optical module 610 may be configured to display different depths-of-field as necessary to see the desired eye anatomies for the different types of ophthalmic surgical procedures. In certain embodiments, each optical module 6100 may be configured to display different resolutions depending on the size of anatomies that are targeted during such surgical procedures. In further embodiments, each optical module 610 may be configured to have different fields of view (e.g. magnification levels), and/or may comprise different light filters, e.g., filters 324 described above.

In still other embodiments, a single optical module 610 comprising two channels 204, wherein each channel 204 has a first lens barrel 208 and second lens barrel 209 working in tandem therein, can be optimized for performing two or more different types of procedures. In such embodiments, the single optical module 610 may be configured to operate within technical specifications, e.g., depths-of-field, resolutions, magnification levels, input light levels, light filters, etc., applicable to each of the two or more different types of procedures.

FIG. 7A illustrates a patient's eye 702 as viewed on a three-dimensional (3D) digital visualization system at different stages of magnification, according to certain embodiments of the present disclosure. As described above, a surgical camera of a 3D digital visualization system may comprise an optical head with a plurality of lens barrels for each of two stereo channels, wherein the optical load for each channel may be split between the plurality of lens barrels. The specific example of FIG. 7A illustrates the separation in optical load for an optical head comprising two lens barrels in each channel, e.g., optical head 200 having optical module 210, as seen by a user on a heads-up display of the corresponding digital visualization system, e.g., heads-up display 106 of digital visualization system 102.

As shown at far left in FIG. 7A, viewing process 700 for viewing ophthalmic anatomies of patient eye 702 may begin at view 710, which comprises a 3D wide field of view (FOV) with no relative magnification (e.g., the image is magnified to a desired “starting” level). View 710 may be provided by a first, wide field lens barrel in each stereo channel, e.g., lens barrels 208 or 408 described above. A user may then magnify or zoom in on eye 702 at a first increment (e.g., 2X) to arrive at view 720 via user adjustment of a digital or physical knob on, e.g., a surgical console in communication with the 3D digital visualization system, or by actuating a foot pedal or other similar control device communicatively coupled to the surgical console. In the example of FIG. 7A, view 720 may be representative of a maximum magnification or zoom level of the first lens barrel. To transition to view 720, a magnification/zoom mechanism for the first lens barrel, such as a digital or optical zoom mechanism, may be triggered by the user's input, thereby magnifying the image provided by the first lens barrel.

Upon reaching the maximum magnification or zoom level of the first lens barrel, the optical head may transition to a second, narrow field lens barrel thereof in each channel, e.g., lens barrels 209 or 409 described above, to arrive at view 730. View 730 may be representative of a 3D narrow FOV as provided by the second lens barrel with no magnification or zoom, and may be substantially the same as view 720 provided by the first lens barrel at its maximum magnification or zoom, thereby making the transition “seamless.” In order to make such a seamless transition between images provided by the two lens barrels, the 3D digital visualization system may crop and rescale images provided by the first, wide field lens barrel as magnification thereof is increased to substantially match the unmagnified image provided by the second, narrow field lens barrel. Additionally, utilizing digital gains and offsets (e.g., digital mechanisms), light levels, color balance, and depth of field of the images are matched, in addition to iris control of the lens barrels.

Returning to FIG. 7A, after transitioning to view 730, upon receiving further input from the user to magnify or zoom in on eye 702, a magnification/zoom mechanism for the second lens barrel, such as a digital or optical zoom mechanism, is triggered to magnify the image provided by the second lens barrel and arrive at final view 740.

FIGS. 7B and 7C illustrate schematic cross-sectional side views of optical head 200 in FIG. 2A, at different stages of adjusting a working distance and/or focus thereof during use, according to certain embodiments of the present disclosure. As previously described, in certain embodiments, the working distance of the optical head 200, and/or the focusing of images captured by optical module 210, may be adjusted by changing the position (e.g., distance) of optical module 210 relative to first mirror-beam splitter module 240 within optical head 200. In such embodiments, optical module 210 may be movably mounted to rail 284 within optical head 200 and laterally translated along rail 284 to adjust the working distance and/or focus of optical head 200 and optical module 210.

To illustrate this concept, FIG. 7B depicts optical module 210 at a first position 750 relative to first mirror-beam splitter module 240, wherein input light 701 travels a distance Z between a mirror or beam splitter 242 of first mirror-beam splitter module 240 and a sensor (e.g., 216 or 218) of lens barrel 208 or 209. In FIG. 7B, optical head 200 has a working distance of Y, which is a function of the total distance traveled by input light 701 between a patient's eye 702 and the sensor of lens barrel 208 or 209, among other things. In FIG. 7C, the optical module 210 is moved a distance X toward first mirror-beam splitter module 240 to a second position 760. Accordingly, in FIG. 7C, the distance traveled by input light 701 between the same mirror or beam splitter 242 of first mirror-beam splitter module 240 and the sensor of lens barrel 208 or 209 is reduced by the distance X (Z−X). Oppositely, the working distance Y is increased by the distance X (Y+X). Thus, laterally translating optical module 210 along rail 284 toward first mirror-beam splitter module 240 can increase a working distance of the optical head 200, and vice versa. Similarly, such translation of the optical module 210 may be utilized to focus images captured by optical module 210. This overall mechanism not only provides a simple means of adjusting working distance and focusing of the optical head 200 and optical module 210, but also helps maintain the relatively compact, (e.g., low-height or low profile) design of optical head 200. Additionally, by moving the entire optical module 210, and thus, all lens barrels and optics together, any tolerance issues associated with individual optic/barrel movement are avoided, and the same resolution is capable of being maintained across different working distances.

In summary, certain embodiments of the present disclosure include improved optical heads for visualization systems, such as heads-up digital surgical visualization systems. The optical heads described herein are optimized for use during multiple different ophthalmic surgical procedures, and which also facilitate improved ergonomics and visibility during ophthalmic procedure. Certain embodiments further provide optical heads with lens barrels that facilitate higher resolution and more efficient use of light, thereby improving patient safety. Such lens barrels may also be disposed in arrangements that reduce the occurrence of optical distortions. Accordingly, the optical systems described herein provide several benefits overall conventional systems.

Although cataract and retinal surgeries are discussed as an example of surgical procedures that may benefit from the described embodiments, the advantages of the surgical devices and systems described herein may benefit other surgical procedures as well.

While the foregoing is directed to embodiments of the present disclosure, other and further embodiments of the disclosure may be devised without departing from the basic scope thereof, and the scope thereof is determined by the claims that follow.

### EXAMPLE EMBODIMENTS

Embodiment 1: A surgical camera, comprising: an outer casing; a baseplate coupled to a lower end of the outer casing, the base plate comprising a window for facilitating ingress and egress of light into the surgical camera; and an optical module coupled to the baseplate and disposed within the outer casing, the optical module comprising: two stereoscopic channels, each stereoscopic channel comprising: a first lens barrel comprising one or more first optics, the first lens barrel configured to produce an image having a wide field of view (FOV); a first sensor coupled to the first lens barrel for receiving the image having the wide FOV; a second lens barrel comprising one or more second optics, the second lens barrel configured to produce an image having a narrow FOV, wherein an optical load of each stereoscopic channel is split between the first lens barrel and the second lens barrel; and, second sensor coupled to the second lens barrel for receiving the image having the narrow FOV.

Embodiment 2: The surgical camera of Embodiment 1, wherein the one or more first optics of each first lens barrel comprise a fixed focal length.

Embodiment 3: The surgical camera of Embodiment 2, wherein the optical module comprises a digital zoom mechanism to magnify the image produced by each first lens barrel.

Embodiment 4: The surgical camera of Embodiment 1, wherein the one or more second optics of each second lens barrel comprise a fixed focal length.

Embodiment 5: The surgical camera of Embodiment 4, wherein the optical module comprises a digital zoom mechanism to magnify the image produced by each second lens barrel.

Embodiment 6: The surgical camera of Embodiment 1, wherein the one or more second optics of each second lens barrel comprise a fixed focal length.

Embodiment 7: The surgical camera of Embodiment 6, wherein the optical module comprises a digital zoom mechanism to magnify the image produced by each first and second lens barrel.

Embodiment 8: The surgical camera of Embodiment 1, wherein the one or more first optics of each first lens barrel comprise movable optics for providing optical zoom.

Embodiment 9: The surgical camera of Embodiment 1, wherein the one or more second optics of each second lens barrel comprise movable optics for providing optical zoom.

Embodiment 10: The surgical camera of Embodiment 1, wherein the first lens barrel and the second lens barrel of each stereoscopic channel are disposed in a side-by-side arrangement in the optical module, and wherein each of the first lens barrel and the second lens barrel are disposed along a different optical axis of the optical module.

Embodiment 11: The surgical camera of Embodiment 1, wherein the first lens barrel and the second lens barrel of each stereoscopic channel are disposed in a stacked arrangement in the optical module, and wherein the first lens barrel and the second lens barrel are disposed along a same optical axis of the optical module.

Embodiment 12: The surgical camera of Embodiment 1, wherein the two stereoscopic channels are disposed in an opposing layout within the optical module such that the first lens barrel and the second lens barrel of one of the two stereoscopic channels are disposed opposite of and in reverse but parallel orientation relative to the first lens barrel and the second lens barrel of the other one of the two stereoscopic channels.

Embodiment 13: The surgical camera of Embodiment 1, wherein the optical module further comprises a mirror-beam splitter module, and wherein input light is split between and directed into the first lens barrel and the second lens barrel of each stereoscopic channel via the mirror-beam splitter module.

Embodiment 14: The surgical camera of Embodiment 1, wherein input light is split between the first lens barrel and the second lens barrel of each stereoscopic channel in a ratio of about 30:70, respectively.

Embodiment 15: The surgical camera of Embodiment 1, wherein each first lens barrel and each second lens barrel of the two stereoscopic channels has an f-number between about f/8 and about f/2.

Embodiment 16: The surgical camera of Embodiment 1, further comprising: a pair of coaxial illumination sources, each of the coaxial illumination sources having an illumination axis coincident with an optical axis of at least one of the first lens barrel and the second lens barrel of each stereoscopic channel.

Embodiment 17: The surgical camera of Embodiment 16, wherein the illumination axis of each coaxial illumination source is coincident with a first optical axis of the first lens barrel in each stereoscopic channel and a second optical axis of the second lens barrel in each stereoscopic channel.

Embodiment 18: The surgical camera of Embodiment 17, wherein the illumination axes, the first optical axes, and the second optical axes are made coincident by one or more mirror-beam splitter modules of the surgical camera.

Embodiment 19: The surgical camera of Embodiment 1, further comprising: a rail anchored to the baseplate, wherein the optical module is movably coupled to the rail; and, an actuator for driving lateral movement of the optical module along the rail.

Embodiment 20: The surgical camera of Embodiment 19, wherein lateral movement of the optical module along the rail facilitates adjustment of a working distance of the surgical camera.

Embodiment 21: The surgical camera of Embodiment 20, wherein lateral movement of the optical module along the rail facilitates adjustment of a focus of the surgical camera.

Embodiment 22: The surgical camera of Embodiment 1, wherein each of the first lens barrels and the second lens barrels comprise one or more irises.

Embodiment 23: The surgical camera of Embodiment 22, wherein opening and closing of the irises is driven by one or more motors.

Embodiment 24: The surgical camera of Embodiment 23, wherein the irises of both first lens barrels are driven by a single motor.

Embodiment 25: The surgical camera of Embodiment 24, wherein the irises of both second lens barrels are driven by a single motor.

Embodiment 26: The surgical camera of Embodiment 1, wherein the optical module is arranged to emulate a straight orientation of the first lens barrels and the second lens barrels.

Embodiment 27: The surgical camera of Embodiment 1, wherein the optical module is arranged to emulate a tilted orientation of the first lens barrels and the second lens barrels.

What is claimed is:

1. An optical system for a surgical camera, the optical system comprising:
two stereoscopic channels, each stereoscopic channel comprising:
a first lens barrel comprising one or more first optics, the first lens barrel configured to produce an image having a wide field of view (FOV); and
a second lens barrel comprising one or more second optics, the second lens barrel configured to produce an image having a narrow FOV, wherein an optical load of each stereoscopic channel is split between the first lens barrel and the second lens barrel.

2. The optical system of claim 1, wherein the one or more first optics of each first lens barrel comprise a fixed focal length.

3. The optical system of claim 2, wherein the optical system comprises a digital zoom mechanism to magnify the image produced by each first lens barrel.

4. The optical system of claim 1, wherein the one or more second optics of each second lens barrel comprise a fixed focal length.

5. The optical system of claim 4, wherein the optical system comprises a digital zoom mechanism to magnify the image produced by each second lens barrel.

6. The optical system of claim 1, wherein the one or more second optics of each second lens barrel comprise a fixed focal length.

7. The optical system of claim 6, wherein the optical system comprises a digital zoom mechanism to magnify the image produced by each first and second lens barrel.

8. The optical system of claim 1, wherein the one or more first optics of each first lens barrel comprise movable optics for providing optical zoom.

9. The optical system of claim 1, wherein the one or more second optics of each second lens barrel comprise movable optics for providing optical zoom.

10. The optical system of claim 1, wherein the first lens barrel and the second lens barrel of each stereoscopic channel are disposed in a side-by-side arrangement, and wherein each of the first lens barrel and the second lens barrel are disposed along a different optical axis of the optical system.

11. The optical system of claim 1, wherein the first lens barrel and the second lens barrel of each stereoscopic channel are disposed in a stacked arrangement, and wherein the first lens barrel and the second lens barrel are disposed along a same optical axis of the optical system.

12. The optical system of claim 1, wherein the two stereoscopic channels are disposed in an opposing layout such that the first lens barrel and the second lens barrel of one of the two stereoscopic channels are disposed opposite of and in reverse but parallel orientation relative to the first lens barrel and the second lens barrel of the other one of the two stereoscopic channels.

13. The optical system of claim 1, wherein input light is split between and directed into the first lens barrel and the second lens barrel of each stereoscopic channel via a beam splitter and mirror.

14. The optical system of claim 1, wherein input light is split between the first lens barrel and the second lens barrel of each stereoscopic channel in a ratio of about 30:70, respectively.

15. The optical system of claim 1, wherein each first lens barrel and each second lens barrel of the two stereoscopic channels has an f-number between about f/8 and about f/2.
